(19) United States
(12) Patent Application Publication
(10) Pub. No.: US 2002/0115635 A1
(43) Pub. Date: Aug. 22, 2002

(54) Modulation of GSK-3beta activity and its different uses

(76) Inventors: Pnina Fishman, Herzliya (IL); Kamel Khalili, Merion, PA (US)

Correspondence Address: BIRCH STEWART KOLASCH & BIRCH, PO BOX 747, FALLS CHURCH, VA, 22040-0747, US

(21) Appl. No.: 09/788,477

(22) Filed: Feb. 21, 2001

(51) Int. Cl. A61K 31/522; A61K 31/52; A61K 31/7076

(52) U.S. Cl. 514/46; 514/263.34; 514/263.4

(57) ABSTRACT

A method for a therapeutic treatment, comprising administering to a subject in need an effective amount of an active agent for achieving a therapeutic effect, the therapeutic effect comprises modulating GSK-3B activity in cells and said active agent is selected from the group consisting of an adenosine A1 receptor ligand (A1RL), an A2 adenosine receptor ligand (A2RL), an adenosine A3 receptor ligand (A3RL) and a combination of the same.

### FIELD OF THE INVENTION

The present invention relates to therapeutic use of adenosine agonists and antagonists.

### BACKGROUND OF THE INVENTION

### Adenosine Receptor Cascade

Adenosine is a ubiquitous nucleoside present in all body cells, It is released from metabolically active or stressed cells and subsequently acts as a regulatory molecule. It binds to cells through specific A1, A_{2A}, A_{2B} and A3 G-protein associated cell surface receptors, thus acting as a signal transduction molecule by regulating the levels of adenylyl cyclase and phospholipase C [Linden J. The FASEB J 5:2668-2676 (1991); Stiles G. L. Clin. Res. 38:10-18 (1990)]. The receptors will be referred to herein after as “A1 receptor” or “A1R”, etc. The binding of adenosine and its agonists to the A3 receptor is known to activate the Gi protein cascade which inhibits adenylate cyclase activity and the production of cAMP.

Recently it was shown that low dose adenosine inhibits in vitro the growth of various humor cells. However, the utilization of adenosine as a therapeutic agent in vivo is restricted, as it rapidly metabolizes and thus, its ability to exert a systemic effect is limited [Fishman P. et al. Cancer Research 58:3181-3187 (1998)].

### The Wnt Signal Transduction Pathway

The Wnt developmental pathway with its most celebrated participants, β-catenin and Lef/Tct has emerged as an important player in a number of neoplasia including malignant melanoma. Wnt's are a family of paracrine and autocrine factors that regulate cell growth and cell fate [Peifer M. and Polakis P. Science 287:1606-1609 (2000)]. Signaling by the Wnt pathway is initiated when Wnt ligands bind to transmembrane receptors of the Frizzled family (FIG. 1). Frizzleds (Frz) signal through Dishevelled (Dsh) to inhibit the kinase activity of a complex containing glycogen synthase kinase 3 (GSK-3β), APC, AXIN and other proteins. The complex targets β-catenin and phosphorylates the threonine and serine residues of exon 3. The phosphorylated β-catenin is rapidly degraded by the ubiquitin-proteasome pathway. Thus, a mutation in the serine and threonine residues of exon 3 of β-catenin prevents phosphorylation of catenin and results in stabilization of the protein. Once hypophosphorylated due to Wnt signaling, stabilized β-catenin accumulates in the cells, translocates to the nucleus where it binds to Lef/Tcf family of transcription factors, upregulates the expression of Wnt target genes including cyclin D and c-myc [Sakanaka C, et al. Recent Prog Horm Res. 55:225-236 (2000)]. In addition, β-catenin is a cytoplasmic protein which associates with cadherin and couples these calcium-dependent membrane-bound adhesion molecules to components of the cytoskeleton [Nollet F. et al. Mol Cell Biol Res Commun 2:77-85 (1999)].

### Wnt Signaling and Human Diseases

Diseases Involved with GSK-3β Deficiency

The involvement of the Wnt pathway in the development of melanoma was first discovered by the presence of a single mutation in the N-terminus of β-catenin [Robbins P F et al. J Exp Med. 183:1185-1192 (1996)]. This discovery was supported by later reports suggesting that downstream components of the Wnt pathway such as APC (adenomaious polyposis coli) and β-catenin, are involved in human cancer. There are also several reports that Wnt ligands are highly expressed in tumors.

In addition there are also reports that defects in the Wnt/APC/β-catenin/Tcf pathway are implicated in other neoplasm. For example, somatic mutations in APC which typically lead to a truncated protein with no regulatory activity can cause the accumulation of free β-catenin. Alternatively, a mutation in β-catenin can increase the half-life of β-catenin, the latter can then stimulate the transcription of cell cycle regulators such as myc and cyclin D. The level of β-catenin could be reduced by overexpression of APC in these cells, and/or enhancement in the activity of GSK-3β which causes phosphorylation of β-catenin and its degradation [Robbins P F et al. J Exp Med. 183:1185-1192 (1996); Barker N et al. Adv Cancer Res. 77:1-24 (2000).

It has also been suggested that enhancement of GSK-3β activity may be therapeutically useful for the treatment of hair loss.

Diseases Involved with GSK-3β Hyper Function

The kinase GSK-3β along with another kinase, cyclin dependent kinase (CDK5) were found to be responsible for some abnormal hyperphosphorylation of the microtubule binding protein tau observed in the neurodegenerative Alzheimer's disease. Thus, agents which inhibit GSK-3β may be useful for the treatment or prevention of not only Alzheimer's disease but also of other hyperphosphorylation related degenerative diseases, such as frontal lobe degeneration, argyrophilic grains disease, and subacute scleroting panencephalitis (as a late complication of viral infection in the central nerve system), and for the treatment of neurotraumatic diseases such as acute stroke, psychiatric (mood) disorders such as schizophrenia and manic depression.

In addition, it has been shown that elevated GSK-3 activity is involved in the development of insulin resistance and type II diabetes (non-insulin dependent diabetes mellitus). Thus, agents which inhibit GSK-3β activity may be used for the treatment or prevention of type II diabetes.

The present invention thus aims for the providence of agents which are capable of modulating the GSK-3β activity. As will be shown in the following description, these agents are adenosine agonists and antagonists.

### SUMMARY OF THE INVENTION

The present invention is based on the surprising finding that ligands of the adenosine receptor are capable of modulating the Wnt signal transduction pathway. Thus, the invention relates in its broadest sense to a method for a therapeutic treatment, comprising administering to a subject in need an effective amount of an active agent for achieving a therapeutic effect, the therapeutic effect comprises modulating GSK-3β0 activity in cells and said active agent is selected from the group consisting of an adenosine A1 receptor ligand (A3RL), A2 receptor ligand (A2RL), A3 receptor ligand (A3RL), and a combination of the same.

The term “ligand” used herein with reference to a specific adenosine receptor (i.e. A1, A2 and A3 receptors) refers to any molecule capable of binding to one or more of the adenosine receptors, thereby influencing the activity of the corresponding receptor (fully or partially). The ligand according to the invention may be specific, e.g. an A1RL is a ligand which specifically binds to the adenosine A1 receptor Alternatively, it may be the case that a ligand binds and modulates the activity of more than one receptor. For example, a ligand may be an adenosine A1 and A3 receptor agonists as well as an A2 receptor antagonist, all of which are known to inhibit adenylate cyclase.

Two main embodiments are provided by the present invention. The first embodiment, to be referred to herein as the “GSK-3β activation embodiment” involves enhancement of the GSK-3β activity in cells, which may have a therapeutic value for the treatment of diseases or disorders associated with GSK-3β deficiently or dysfunction As indicated hereinbefore, it has been described that cancer is associated with GSK-3β deficiency. Thus, agents which are capable of enhancing GSK-3β activity may be of therapeutic use in the treatment or prevention of diseases or disorders associated with abnormal cell proliferation. To this end, the present invention provides agents which enhance this kinase's activity. These agents are, in general, adenosine receptor ligands selected from the group consisting of adenosine A1 receptor agonist (A1RAg), adenosine A3 receptor agonist (A3RAg), adenosine A2 receptor antagonist (A2RAn) and any combination of A1RAg, A3RAg and A2RAn.

The second embodiment of the present invention, to be referred to herein as the “GSK-3β inhibition embodiment” involves reduction/suppression of the kinase activity, which, accordingly, may have a therapeutic value for the treatment of diseases or disorders associated with GSK-3β elevated activity. As indicated hereinbefore, there are several illnesses which result from hyperphosphorylation by this kinase. Thus, agents capable of suppressing GSK-3β activity may have therapeutic use in the treatment or prevention of such illnesses. To this end, the present invention provides agents which inhibit GSK-3β activity. These biologically active agents are adenosine receptor ligands selected from the group consisting of adenosine A1 receptor antagonist (A1RAn), adenosine A3 receptor antagonist (A3RAn), adenosine A2 receptor agonist (A2RAg) and any combination of A1RAn, A3RAn and A2RAg.

The term “treatment” as used herein refers to the administering of a therapeutic effective amount of the agent provided by the present invention, the amount being sufficient to achieve a therapeutic effect leading to amelioration of undesired symptoms associated with a disease such as those described above (e.g. hair loss, Alzheimer's disease. acute stroke, schizophrenia, manic depression, etc.), prevention of the manifestation of such symptoms before they occur, slowing down the deterioration of the symptoms, slowing down the progression of the disease, lessening the severity or cuing the disease, improving of the survival rate or more rapid recovery of a the subject suffering from the disease, prevention of the disease form occurring or a combination of two or more of the above.

The “effective amount ” for purposes herein is determined by such considerations as may be known in the art. The amount must be effective to achieve the desired therapeutic effect as described above, i.e. modulation of GSK-3β, depending, inter alia, on the type and severity of the disease to be treated and the treatment regime. The person versed in the art will know how to determined the effective amount.

The present invention also provides pharimaceutical compositions for achieving a therapeutic effect in a subject in need, the therapeutic effect comprising modulating GSK-3β activity in target cells, the compositions comprising an effective amount of an active agent and one or more pharmaceutically acceptable additives, the active agent is selected from the group consisting of an A1RL, an A2RL, an A3RL and a combination of the same.

The term “target cells” for purposed used herein refers to cells in which the level of GSK-3β is abnormal, i.e. elevated or reduces as compared to the level of GSK-3β is these cells under normal conditions (a non-diseased state) and where modulation of the GSK-3β level in these cells provides treatment, in a subject in need of such treatment, for a disease associated (directly or indirectly) with said abnormal level of GSK-3β.

It should be understood that the present invention provides pharmaceutical compositions for both embodiments of the invention as disclosed herein. Thus, in accordance with the first embodiment, i.e. the “GSK-3β activation embodiment”, the composition of the invention will comprise one or more agents capable of elevating GSK-3β activity in cells. As disclosed above, such agents include the A1RAg, A3RAg, A2RAn and any combination of the same.

Alternatively, the composition of the invention may form part of the “GSK-3β inhibition embodiment”, which accordingly comprises one or more agents capable of suppressing GSK-3β activity in cells, being selected from the group consisting of A1RAn, A3RAn, A2RAg and any combination of the same.

Evidently, any other use of the above described active agents in association with modulation of GSK-3β activity in cells, preferably target cells, either for inhibiting or elevating its activity, will form part of the present invention.

### BRIEF DESCRIPTION OF THE DRAWINGS

In order to understand the invention and to see how it may be cared out in practice, a preferred embodiment will now be described, by way of non-limiting example only, with reference to the accompanying drawings, in which:

FIG. 1 is a schematic illustration of the Wnt signaling pathway.

FIG. 2 is a Western blot analysis of protein extracts from untreated (control in left lane) and Cl-IB-MECA-treated (right lane) melanoma cells using anti-β-catenin antibody.

FIG. 3 is an immunohistochemistry test depicting the level of GSK-3β in melanoma cells upon CI-IB-MECA treatment showing high levels of GSK3-β in the Cl-IB-MECA treated cells.

FIG. 4 is an immunohistochemistry test depicting the level of β-catenin in untreated and Cl-IB-MECA treated cells.

FIG. 5 is an immunohistochemistry test depicting the level of Lef/Tcf in melanoma cells upon Cl-IB-MECA treatment, where Lef/Tcf level is decreased in the Cl-IB-MECA treated cells.

FIG. 6 is a Western blot analysis of the level of Cyclin D1 after modulation of samples with Cl-IB-MECA.

FIG. 7 is a Western blot analysis of the level of cyclin D1 after modulation with Cl-IB-MECA. Using anti-Cyclin D1 antibody, a prominent lane was detected in the sample of untreated mice (left lane “Contorl”), representing the level of Cyclin D1, while in the treated group, a decreased level of Cyclin D1 is seen (right lane, “Cl-IB-MECA”).

FIG. 8 is a Western blot analysis of protein extracts from tumor tissue derived from colon carcinoma bearing mice (treated and untreated with Cl-IB-MECA). Using anti-β-catenin antibody, a prominent lane, representing the level of β-catenin, was detected in the sample of untreated mice (left lane “Control”), while with the treated mice, a decreased level of β-catenin is observed (right lane, “Cl-IB-MECA”)

FIG. 9 is a Western blot analysis of protein extracts from tumor tissue derived from colon carcinoma bearing mice (treated and untreated with Cl-IB-MECA). Using anti-c-myc antibody, a prominent lane, representing the level of c-myc, was detected in the sample of untreated mice (left lane “Control”), while with the treated mice, a decreased level of c-myc is observed (right lane, “Cl-IB-MECA”)

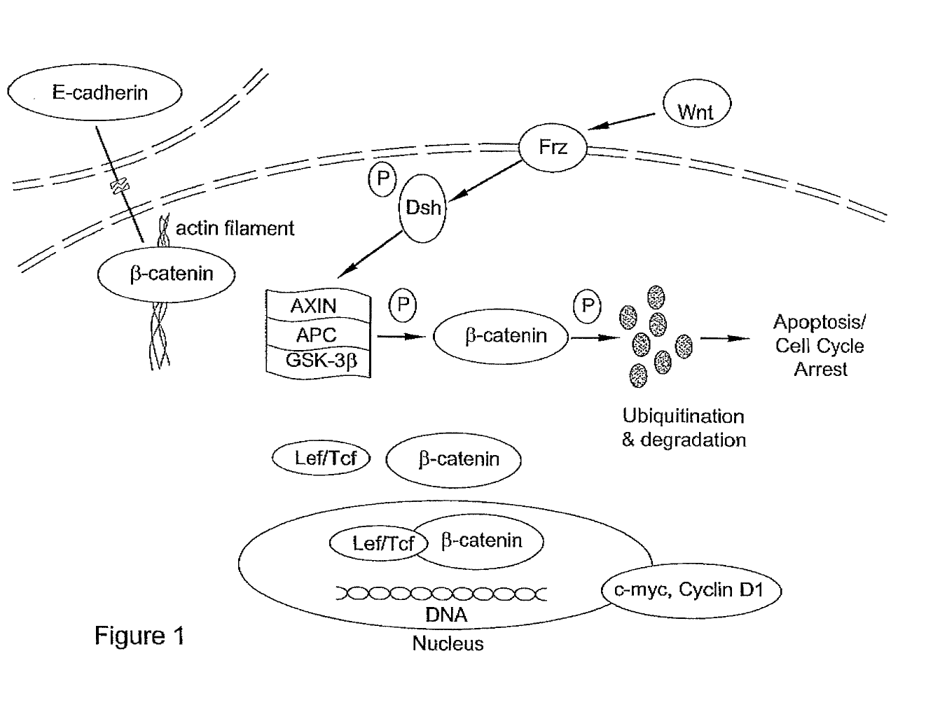

### DETAILED DESCRIPTION OF THE INVENTION

As will be shown in the following specific Examples an increased level of GSK-3β with a decreased β-catenin and Lef/Tcf levels were found following treatment of the B-16 melanoma cells with Cl-IB-MECA as well as a decrease in the level of cyclin D1, one of the end products of the Wnt pathway and a key elements of cell cycle progression.

It was thus suggested by the inventors of the present invention that molecules involved in the signaling pathway associated with Gi protein receptors (i.e. adenosine receptors) may also play a role in modulation of GSK-3β.

Thus, and as disclosed hereinbefore, the present invention provides a method for a therapeutic treatment comprising administering to a subject in need an effective amount of an active agent for achieving a therapeutic effect, the therapeutic effect comprises modulating GSK-3β activity in cells and said active agent is selected from the group consisting of an adenosine A1 receptor ligand (A3RL), A2 receptor ligand (A2RL), A3 receptor ligand (A3RL), and a combination of the same.

In the case of the GSK-3β activation embodiment of the present invention, the agents are adenosine receptor ligands selected from the group consisting of adenosine A1 receptor agonist (A1RAg), adenosine A3 receptor agonist (A3RAg), adenosine A2 receptor antagonist (A2RAn) and any combination of A1RAg, A3RAg and A2RAn.

Some of the agents of the present invention and their synthesis procedure may be found in detail in U.S. Pat. No. 5,688,774; U.S. Pat. No. 5,773,423, U.S. Pat. No. 5,573,772, U.S. Pat. No. 5,443,836, U.S. Pat. No. 6,048,865, WO 95/02604, WO 99120284 and WO 99/06053, WO 97/27173, incorporated herein by reference.

According to one aspect forming part of this GSK-3β activation embodiment, the active agent is an A1RAg. Non-limiting examples of such agents include N⁶-cyclopentyl adenosine (CPA), 2-chloro-CPA (CCPA), N⁶-cyclohexyl adenosine (CHA), N6-(phenyl-2R-isopropyl)adenosine (R-PIA) and 8-{4-[({[(2-aminocthyl)amino]carbonyl}methyl)oxyl-phenyl}-1,3-dipropylxanthine (XAC).

According to another aspect forming part of the GSK-3β activation embodiment!t the active agent is an A3RAg. Non-limiting examples of such agents include 2-(4-aminophenyl)ethyladenosine (APNEA), N⁶-(4-amino-3-iodobenzyl) adenosine-5′-(N-methyluronamide) (AB-MECA) and 1-deoxy-1-{6- [({3-iodophenyl}methyl)amino]-9H-purine-9-yl}-N-methyl-β-D-ribofuranuron-amide (IB-MECA) and preferably 2-chloro-N⁶-(2-iodobenzyl)-adenosine-5′-N-methly-uronamide (Cl-IB-MECA). Other A3RAg include N⁶-benzyl-adenosine-5′-alkyluronamidc-N¹-oxide or N⁶-benzyladenosine-5′-N-dialyluron-amide-N¹-oxide

Yet further, the active agent forming part of the GSK-3β activation embodiment may be an A2RAn. A non-limiting example include 3,7-dimethyl-1-propargyl-xantane (DMPX).

Notwithstanding the above, when referring to the GSK-3β inhibition embodiment, the agents are adenosine receptor ligands selected from the group consisting of adenosine A1 receptor antagonist (A1RAn), adenosine A3 receptor antagonist (A3RAn), adenosine A2 receptor agonist (A2RAg) and any combination of A1RAn, A3RAn and A2RAg.

According to one aspect forming part of the GSK-3β inhibition embodiment, the active agent is an A1RAn. A non-limiting example of such an agent includes 1,3-dipropyl-8-cyclopentylxanthine (DPCPX).

According to a further aspect forming part of this embodiment the active agent is an A3RAn. Non-limiting examples of such agents include 5-propyl-2-ethyl-4-propyl-3-ethylsulfanylcarbonyl)-6-phenylpyridine-5-carboxylate (MRS-1523) and 9-chloro-2-(2-furanyl)-5-[(phenylacetyl)amino] [1,2,4,]-triazolo[1,5-c] quinazoline (MRS-1200).

Yet further A2RAg may be selected as an agent for use in the GSK-3β inhibition embodiment. Such an agent may be, without being limited thereto N⁶-[2-(3,5-dimethoxyphenyl)-2-(2-methylphenyl)-ethyl] adenosine (DMPA).

The active agents disclosed herein may be is administered and dosed in accordance with good medical practice, taking into account the clinical condition of the individual patient, the site and method of administration, scheduling of administration, patient age, sex, body weight and other factors blown to medical practitioners. Accordingly, the active agent may be administered orally, subcutancously or parenterally including intravenous, intraaterial, intramuscular, intraperitoneally and intranasal administration as well as by infusion techniques. However, oral administration is preferable.

For achieving the desired therapeutic effect the active agent may be administered as the low molecular weight compound or as a pharmaceutically acceptable salt thereof and can be administered alone in combination with pharmaceutically acceptable additives. Accordingly, the present invention also provides pharmaceutical compositions for achieving a therapeutic effect in a subject in need, the therapeutic effect comprising modulating GSK-3β activity in cells, the composition comprising a therapeutically effective amount of one or more active agents and one or more pharmaceutically acceptable additives, said active agent is selected from the group consisting of an adenosine A1 receptor ligand (A1RL), an A2 adenosine receptor ligand (A2RL), an adenosine A3 receptor ligand and any combination of A1RL, A2RL and A3RL.

The term “pharmaceutically acceptable additives” used herein refers to any substance combined with said active agent and include, without being limited thereto, diluents, excipients, carriers, solid or liquid fillers or encapsulating materials which are typically added to formulations to give them a form or consistency when it is given in a specific form, e.g. in pill form, as a simple syrup, aromatic powder, and other various elixirs. The additives may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc.

Preferably, the additives are inert, non-toxic materials, which do not react with the active ingredient of the invention. Yet, the additives may be designed to enhance the binding of the active agent to its receptor. Further, the term additive may also include adjuvants, which, by definition, are substances affecting the action of the active ingredient in a predictable way.

The additive can be any of those conventionally used and is limited only by chemico-physical considerations, such as solubility and lack of reactivity with the compound, and by the route of administration.

It is noted that humans are treated generally longer than experimental animals as exemplified herein, which treatment has a length proportional to the length of the disease process and active agent effectiveness. The doses may be single doses or multiple doses over a period of several days. The treatment generally has a length proportional to the length of the disease process and active agent effectiveness and the patient species being treated.

The active agent of the invention may be administered orally to the patient. Conventional methods such as administering the active agent in tablets, suspensions, solutions, emulsions, capsules, powders, syrups and the like are usable.

For oral administration, the composition of the invention may contain additives for facilitating oral delivery of the active agent. Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch Tablet forms can include one or more of lactose, sucrose, mannitol, corn starch, potato starch, alginic acid, microcrystalline cellulose, acacia, gelatin, guar gum, colloidal silicon dioxide, croscarmellose sodiumk talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active agent in a flavor, usually sucrose and acacia or tragacanth, as well as pastilles comprising the active ingredient in an inert base, such as gelatin and glycerin, or sucrose and acacia, emulsions, gels, and the like. Such additives are known in the art.

Alternatively, the active agent may be administered to the patient parenterally In this case, the composition will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof and vegetable oils.

Proper fluidity can be maintained, for example, by the use of a coating such as lecithin, by the maintenance of the required particle size in the case of dispersion and by the use of surfactants. Non-aqueous vehicles such as cottonseed oil, sesame oil, olive oil. soybean oil, corn oil, sunflower oil, or peanut oil and ester, such as isopropyl myristate, may also be used as solvent systems for the composition of the present invention.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammnonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamnides, and polyoxy-ethylenepolypropylene copolyners, (d) amphoteric detergents such as, for example, alkyl-β-aminopriopionates, and 2 -alkyl-imidazoline quaternary ammonium salts, and (3) mixtures thereof

Further, in order to minimize or eliminate irritation at the site of injection, the compositions may contain one or more nonionic surfactants having a hydrophile-lipophile balance (HLB) of from about 12 to about 17. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Obviously, many modifications and variations of the present invention are possible in light of the above teaching. Accordingly, it should be understood that any other use of modulation, by adenosine receptor ligands, of GSK-3β activity in cells which is within the scope of the appended claims forms part of the present invention and that the invention may be practiced otherwise than as specifically described hereinafter.

### SPECIFIC EXAMPLES

### Materials and Methods

B-16-F10 murine melanoma cell line was employed in the following in vitro and ex vivo experiments. Cells were maintained in RPMI medium containing 10% FBS, penicillin and streptomycin. They were transferred twice weekly to a freshly prepared medium.

B-16 F10 melanoma cells (10⁶) were cultured in 24-well plates in the presence and absence of 10 nM Cl-IB-MECA for 24 hours.

To analyze key elements in the Wnt pathway, several methods were used, in including (1) immunohistological staining (utilizing stained cells), (2) Western blot (utilizing protein extract from the tested cells), and (3) ribonuclease protection assay (RPA) (utilizing RNA extracted from the cells).

### Example 1

### The Effect of Cl-IB-MECA on the Wnt Pathway in B-16 Melanoma Cells

The effect of the A3AR agonist Cl-IB-MECA, on the Wnt signaling pathway in B-16 melanoma cells was evaluated, In particular, the levels of expression of key members of the Wnt pathway, including β-catenin, GSK-3β, Lef/Tcf and of the genes cyclin D1 and c-myc, responsive to Wnt regulation, were determined by Western blot analysis of protein extracts derived from tumor cells at various stages and by immunocytological staining.

### Immunohistological Staining

Immunohistological staining of Cl-IB-MECA-treated or untreated B-16-F10 melanoma cell specimens performed according to the following protocol:

Cells were cultured on Poly-L-Lysine coated glass chamber slides, until they reached approximately 90% confluence, after which they were washed with PBS and fixed with cold acetone for three minutes.

Immunocytochemistry was performed by using the fluorescent system, according to the manufacturer's instructions (Immunofluorescence Kit, Vector Laboratories). In general, slides were rinsed with PBS and blocked in 0-1% BSA in PBS containing 5% normal horse- or goat-serum for 2 hours at room temperature. Then cells were incubated with a primary antibody overnight at room temperature in a humidified chamber. The antibodies used are rabbit polyclonal antibody against GSK-3β (Santa Cruz Biotechnology Inc.), goat polyclonal antibody against LEF-1 (Santa Cruz Biotechnology Inc.), goat polyclonal antibody against TCF-4 (Santa Cruz Biotechnology Inc.) and goat polyclonal antibody against β-Catenin (Santa CruzBiotechnology Inc.).

After rinsing with PBS, secondary FITC conjugated antibodies were incubated at room temperature for 1 hour in tie dark. Then, the cells were rinsed again with PBS, chambers were removed and slides were cover-slipped with an aqueous mounting media. Pictures were taken with an Ultraviolet microscope using a FITC cube and with a phase filter.

### Western Blot Assay

Protein from the Cl-IB-MECA treated or untreated B-16 melanoma cells was extracted for determining β-catenin expression. In particular, the protein extract was separated on gel electrophoresis and then blots on a nitrocellulose membrane. The specific protein was detected by its binding to a specific antibody, in this case, to monoclonal anti-β-catenin antibody.

### Ribonuclease Protection Assay (RPA)

RPA was carried out to examine the level of expression and activity of cyclin D1,D2 and D3 using the multi probe RPA system. In this assay RNA was extracted from Cl-IB-MECA treated or untreated B-16 melanoma cells. The assay kit enabled the generation of a series of templates each of distinct length and cach representing a sequence in a distinct mRNA species. The probe set was hybridized in excess to target RNA in solution, after which free probe and other single-stranded RNA were digested with RNAases, The remaining “Rnases-protected” probes were purified, resolved denaturing polyacrylamide gels, and quantified by phosphor-imaging. The quantity of each mRNA in the original RNA sample was then determined based on the intensity of the appropriately-sized, protected probe fragment.

### Results

Cl-IB-MECA Alters β-Catenin Expression in Melanoma Cells

Several lines of study strongly suggest that β-catenin is a key component of the Wnt signaling pathway which modulates expression of cell cycle genes such as cyclin D and c-myc, plays an important role in the development of melanoma and other neoplastic cells. Free β-catenin, an unstable cytoplasmic protein in normal cells, becomes more stable in neoplastic cells. The free β-catenin migrates to the nucleus and by associating with Lef/Tcf, it stimulates transcription of cyclin D1 and c-myc. In normal cells, phosphorylation of β-catenin by glycogen synthase kinase (GSK) mediates its degradation in the cytoplasm.

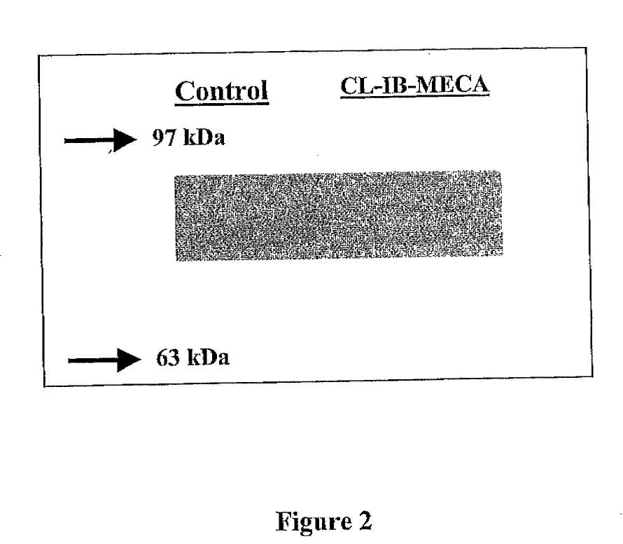

The level of free β-catenin in the untreated and Cl-IB-MECA-treated B16-F10 melanoma cells by Western blot assay was examined. As shown in FIG. 2, while a distinct band corresponding to β-catenin was detected in untreated cells, no band corresponding to β-catenin was detected in the treated cells suggesting that β-catenin is rapidly degraded in the Cl-IB-MECA treated cells.

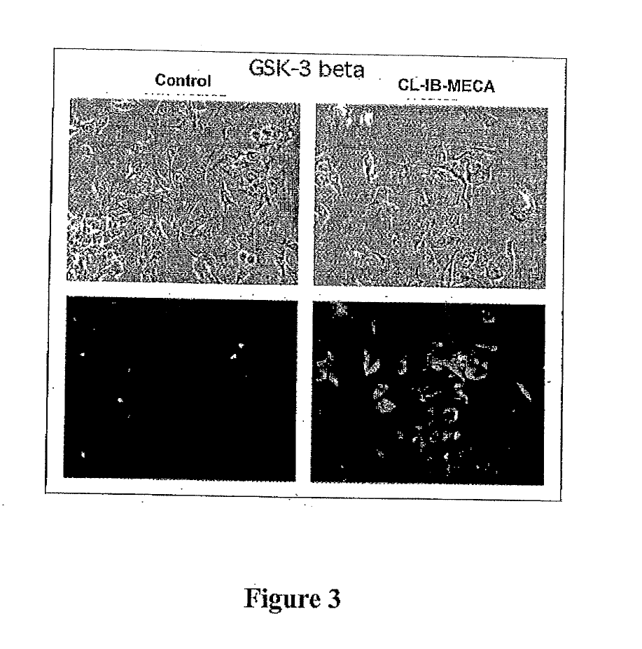

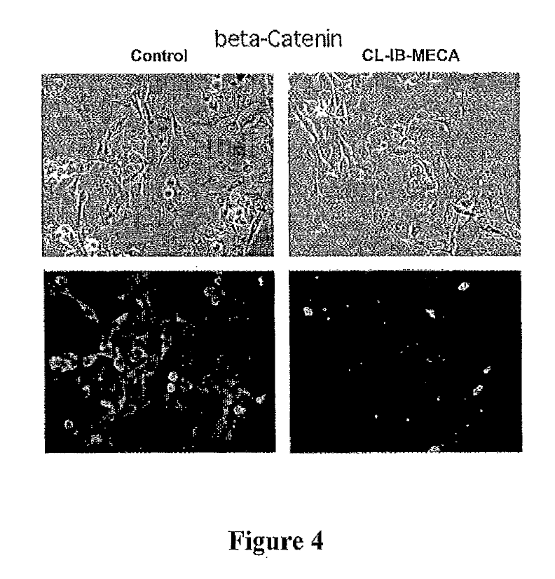

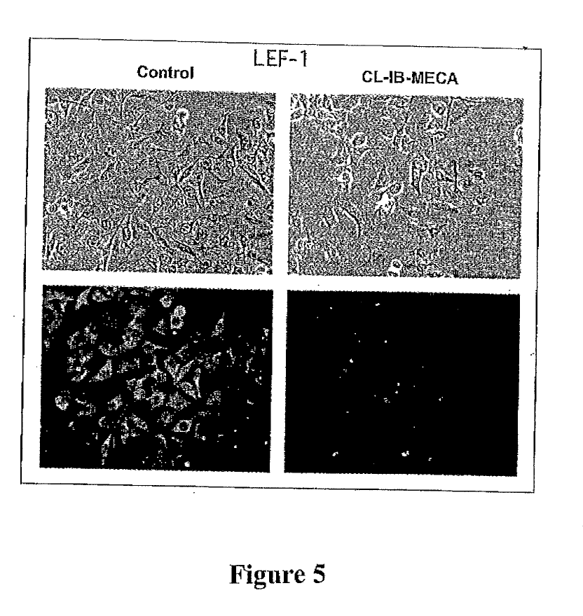

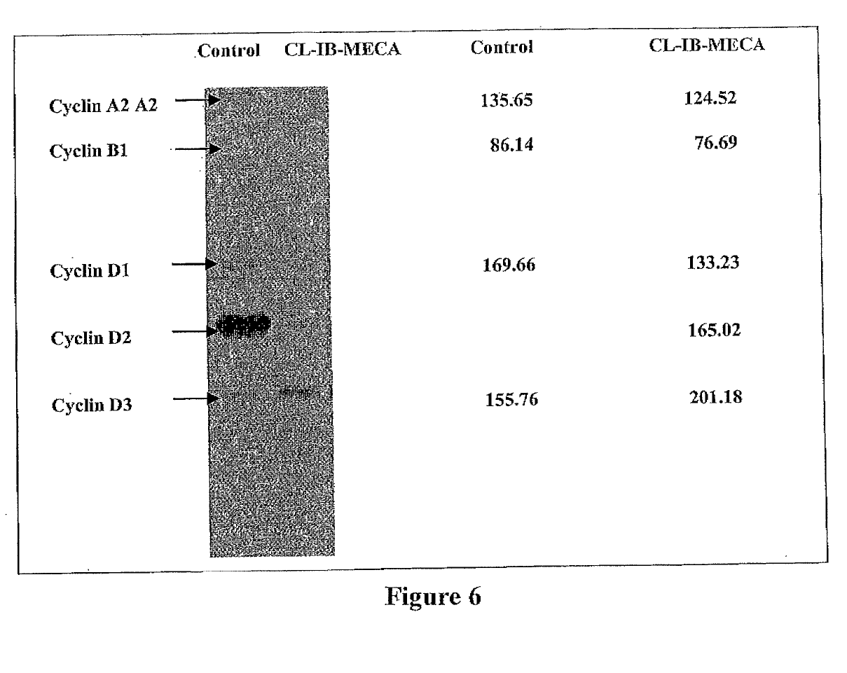

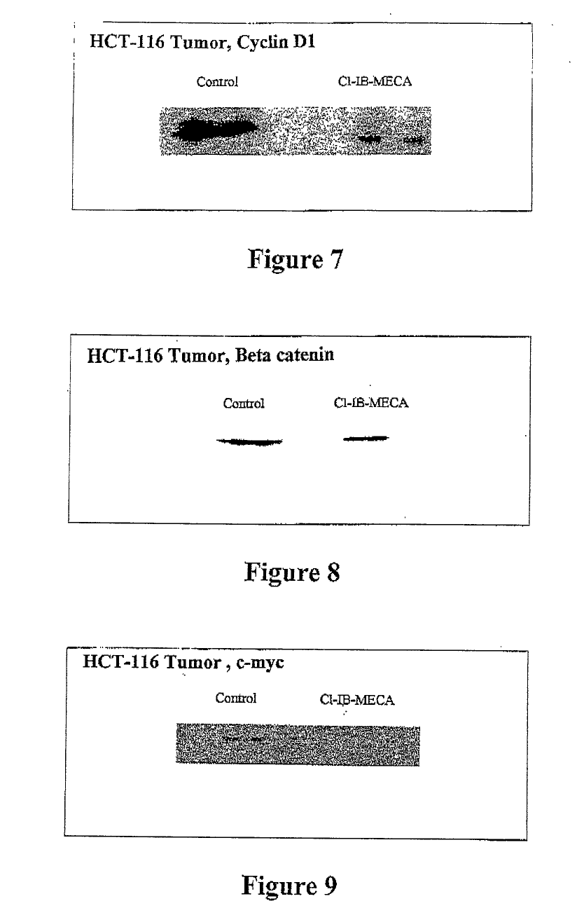

While the mechanistic event loading to the rapid degradation of β-catenin in the treated cells at present remains unclear, one may speculate that enhanced expression and/or activity of GSK-3β which phosphorylates β-catenin may contribute to this event. In support of this concept results from immunocytological staining of treated and untreated cells with anti-GSK-3β revealed enhanced levels of GSK-3β in the treated but not untreated B16-F10 melanoma cells (FIG. 3). Accordingly, in corroboration with the Western blot data, results from immunocytological showed high level of cytoplasmic and nuclear staining of β-catenin in untreated cells, but none in the treated cells (FIG. 4). Moreover the level of Lef/Tcf was found to be decreased following Cl-IB-MECA treatment (FIG. 5), Using the RPA system the level of Cyclin D1 was analyzed (FIG. 6). FIG. 6 shows that the level of Cyclin D1was decreased in the Cl-IB-MECA treated samples, while Cyclin D1 was over expressed in tumor cells leading to un-controlled cell proliferation.

### Example 2

### The Effect of Cl-IB-MECA on the Wnt Pathway in Colon Carcinoma Bearing Mice

A similar series of studies were utilized in a colon carcinoma murine animal model to determine whether elements of the Wnt pathway altered by Cl-IB-MECA in vitro, occur in vivo as well. The animal model was generated by subcutaneous injection of 1.2×10⁶ HCT-116 human colon carcinoma cells to the flank of Balb/C nude mice. The mice were treated orally (by gavage), every second day with 6 μg/Kg Cl-IB-MECA.

After 30 days the mice were sacrificed and tissue samples from the colon carcinoma foci were harvested and analyzed for tie expression of β-catenin and cyclin D1as described above.

### Results

To support the results of the in vitro experiments described above, a similar study was performed tumor tissue derived from mice inoculated with human colon carcinoma HCT-116 cells.

FIG. 6, 7 and. 8 show that Western blot analysis from protein extracts of tumor tissue, derived from Cl-IB-MECA treated and untreated mice, resulted in a decrease in the level of β-catenin, cyclin D1 and c-myc which is in agreement with the in vitro results.

### Conclusion

The above described results provide evidence for the participation of the Wnt signaling pathway in Cl-IB-MECA mediated melanoma and colon carcinoma cell growth in vitro.

It may thus be concluded that Cl-IB-MECA induces the following events; activation of GSK-3β with a subsequent phosphorylation of β-catenin, leading to its degradation. This prevented the migration of β-catenin to the nucleus and the induction of cyclin D1 expression, thus leading to a cell cycle arrest.

1. A method for a therapeutic treatment, comprising administering to a subject in need an effective amount of an active agent for achieving a therapeutic effect, the therapeutic effect conprises modulating GSK-3β activity in cells and said active agent is selected from the group consisting of an adenosine A1 receptor ligand (A1RL) and A2 adenosine receptor ligand (A2RL), an adenosine A3 receptor ligand (A3RL) and a combination of the same.

2. The method of claim 1, wherein said modulation involves activation of GSK-3β activity and said agent is selected from the group consisting of an adenosine A1 receptor agonist (A1RAg), an adenosine A3 receptor agonist (A3RAg), an adenosine A2 receptor antagonist (A2RAn) and a combination of the same.

3. The method of claim 1, wherein said modulation involves inhibition of GSK-3β activity and said agent is selected from the group consisting of an adenosine A1 receptor antagonist (A1RAn), an adenosine A3 receptor agonist (A3RAn), an adenosine A2 receptor agonist (A2RAg) and a combination of the same.

4. The method of claim 2, wherein said active agent is A1RAg.

5. The method of claim 5, wherein said A1RAg is selected from the group consisting of N⁶-cyclopenyl adenosine (CPA), 2-chloro-CPA (CCPA), N⁶-cyclohexyl adenosine (CHA), N6-(phenyl-2R-isopropyl)adenosine (R-PIA) and 8-{4-[({[(2-aminoethyl)amino]carbonyl}methyl)oxyl-phenyl}-1,3-dipropylxanthine (XAC).

6. The method of claim 2, wherein said active agent is an adenosine A1 receptor agonist (A3RAg).

7. The method of claim 6, wherein said A3RAg is selected from the group consisting group consisting of 2-(4-aminophenyl)ethyladenosine (APNEA), N⁶-(4-amino-3-iodobenzyl) adenosine-5′-(N-methyluronamide) (AB-MECA) and 1-deoxy-1-{6-[({3-iodophenyl} methyl)amino]-9H-purine-9-yl}-N-methyl-β-D-ribofuranuron-amide (IB-MECA) and 2-chloro-N⁶-(2-iodobenzyl)-adenosine-5′-N-methly-uronamide (Cl-IB-MECA).

8. The method of claim 6, wherein the active agent is CI-IB-MECA.

9. The method of claim 6, wherein the active agent is a xanthine-7-riboside derivative.

10. The method of claim 2 wherein said active agent is an adenosine A2 receptor antagonist (A2RAn).

11. The method of claim 10, wherein said A2RAn is 3,7-dimethyl-1-propargyl-xantane (DMPX).

12. The method of claim 2, for the treatment of a disease or disorder which requires for its treatment elevation of GSK-3β activity.

13. The method of claim 12 wherein said disease is hair loss.

14. The method of claim 3, wherein said active agent is an A1RAn.

15. The method of claim 14, wherein said A1RAn is 1,3-dipropyl-8-cyclopentylxanthine (DPCPX).

16. The method of claim 3, wherein said active agent is an A3RAn.

17. The method of claim 16, wherein said A3RAn is selected from the group consisting of 5-propyl-2-ethyl-4-propyl-3-ethysulfanylcarbonyl)-6-phenylpyridine-5-carboxylate (MRS-1523) and 9-chloro-2-(2-furanyl)-5[(phenylacetyl)amino][1,2,4,]-triazolo[1,5-c]quinazoline (MRS-1200):

18. The method of claim 3, wherein said active agent is an adenosine A2RAg.

19. The method of claim 18, wherein said A2RAg is N⁶-[2-(3,5-dimethoxyphenyl)-2-(2-methylphenyl)-ethyl] adenosine (DMPA)

20. The method of claim 3, for the treatment of a disease or disorder which requires for its treatment suppression of GSK-3β activity.

21. The method of claim 20, wherein said disease is a disease associated with degeneration of cells.

22. The method of claim 20, wherein said disease is a neurodegenerative disease or a neurotraumatic disorder.

23. The method of claim 20, wherein said disorder is associated with psychiatric disorders.

24. The method of claim 20, wherein said disease is non-insulin dependent diabetes mellitus.

25. The method of claim 1, wherein said active agent is administered orally.

26. A pharmaceutical composition for achieving a therapeutic effect in a subject in need, the therapeutic effect comprising modulating GSK-3β activity in target cells, the composition comprising a therapeutically effective amount of an active agent and one or more pharmaceutically acceptable additives, said active agent is selected from the group consisting of an adenosine A1 receptor ligand (A1RL), an A2 adenosine receptor ligand (A2RL), an adenosine A3 receptor ligand and any combination of A1RL, A2RL and A3RL.

27. The composition of claim 26, wherein said modulation involves activation of GSK-3β activity and said agent is selected from the group consisting of an adenosine A1 receptor agonist (A1RAg), an adenosine A3 receptor agonist (A3RAg), an adenosine A2 receptor antagonist (A2RAn) and a combination of the same.

28. The composition of claim 26, wherein said modulation is inhibition of GSK-3β activity and said agent is selected from the group consisting of an adenosine A1 receptor antagonist (A1RAn), an adenosine A3 receptor antagonist (A3RAn), an adenosine A2 receptor agonist (A2RAg) and a combination of the same.

29. The composition of claim 27, wherein said active agent is A1RAg.

30. The composition of claim 29, wherein said A1RAg is selected from the group consisting of the N⁶-cyclopentyl adenosine (CPA), 2-chloro-CPA (CCPA), N⁶-cyclohexyl adenosine (CHA), N6-phenyl-2R-isopropyl)adenosine (R-PIA) and 8-{4-[({[(2-aminoethyl)amino]carbonyl}methyl)oxyl-phenyl}-1,3-dipropylxanthine (XAC).

31. The composition of claim 27, wherein said active agent is an adenosine A3 receptor agonist (A3RAg).

32. The composition of claim 31, wherein said A3RAg is selected from the group consisting group consisting of 2-(4-aminophenyl)ethyladenosine (APNEA), N⁶-(4-amino-3- iodobenzyl) adenosine-5′-(N-methyluronamide) (AB-MECA) and 1-deoxy-1-{6- [({3-iodophenyl} methyl)amino]-9H-purine-9-yl}-N-methyl-β-D-ribofuranuron-amide (IB-MECA) and 2-chloro-N⁶-(2-iodobenzyl)-adenosine-5′-N-methly-uronamide (Cl-IB-MECA).

33. The composition of claim 32, wherein the active agent is Cl-IB-MECA.

34. The composition of claim 31, wherein the active agent is a xanthine-7-riboside derivative.

35. The composition of claim 27, wherein said active agent is an adenosine A2 receptor antagonist (A2RAn).

36. The composition of claim 35, wherein said A2RAn is 3,7-dimethyl-1-propargyl-xantane (DMPX).

37. The composition of claim 27, for the treatment of a disease or disorder which requires for its treatment elevation of GSK-3β activity.

38. The composition of claim 37, for the treatment of hair loss.

39. The composition of claim 28, wherein said active agent is an A3RAn.

40. The composition of claim 39, wherein said A3RAn is 5-propyl-2-ethyl-4propyl-3-ethylsulfanylcarbonyl)-6-phenylpyridine-5-carboxylate (MRS-1523) and 9-chloro-2-(2-furanyl)-5-[(phenylacetyl)amino][1,2,4,]-triazolo[1,5-c]quinazoline (MRS-1200)

41. The composition of claim 28, wherein said active agent is an A1RAn.

42. The composition of claim 41, wherein said A1RAn is 1,3-dipropyl-8-cyclopentylxanthine (DPCPX).

43. The composition of claim 28, wherein said active agent is an A2RAg.

44. The composition of claim 43, wherein said A2RAg is N⁶-[2-(3,5-dimethoxyphenyl)-2 -(methylphenyl)-ethyl ]adenosine (DMPA).

45. The composition of claim 26, for the treatment of a disease or disorder which requires for its treatment suppression of GSK-3β activity.

46. The composition of claim 45, wherein said disease is a disease associated with degeneration of cells.

47. The composition of claim 45, wherein said disease is a neurodegenerative disease or a neurotraumatic disorder.

48. The composition of claim 45, wherein said disorder is associated with psychiatric disorders.

49. The composition of claim 45, wherein said disease is non-insulin dependent diabetes mellitus.

50. The composition of claim 26, formulated for oral administration.

51. Use of an active agent selected from the group consisting of an adenosine A1 receptor ligand (A1RL), an adenosine A2 receptor ligand (A2RL), and an adenosine A3 receptor ligand (A3RL) and any combination of A1RL, A2RL and A3RL for modulating GSK-3β activity in cells.

52. Use according to claim 51, for elevating GSK-3β activity, wherein said active agent is selected from the group consisting of A1RAg, A3RAg, A2RAn and any combination of the same.

53. Use according to claim 51, for suppressing GSK-3β activity, wherein said active agent is selected from the group consisting of A1RAn, A3RAn, A2RAg and any combination of the same.

54. Use according to Claim 51, substantially as described in the specification.
